(19) United States
(12) Patent Application Publication
(10) Pub. No.: US 2025/0034065 A1
(43) Pub. Date: Jan. 30, 2025

(54) DEVICE AND PROCESS FOR CONVERTING AROMATICS HAVING 9 CARBON ATOMS

(71) Applicant: IFP ENERGIES NOUVELLES, Rueil-Malmaison (FR)

(72) Inventors: Christophe BOUCHY, RUEIL-MALMAISON CEDEX (FR); Vincent COUPARD, RUEIL-MALMAISON CEDEX (FR); Alexandre PAGOT, RUEIL-MALMAISON CEDEX (FR); Thi Bich Ngoc DANG, RUEIL-MALMAISON CEDEX (FR)

(73) Assignee: IFP ENERGIES NOUVELLES, Rueil-Malmaison (FR)

(21) Appl. No.: 18/716,743

(22) PCT Filed: Nov. 26, 2022
(86) PCT No.: PCT/EP2022/083380

§ 371 Date: Jun. 5, 2024

(30) Foreign Application Priority Data
Dec. 6, 2021 (FR) 2113010

(51) Int. Cl. C07C 5/27

(52) U.S. Cl. CPC C07C 5/2791; C07C 2529/74

(57) ABSTRACT

The present invention relates to a process and a device for the conversion of aromatic compounds, in which the aromatic compounds of a hydrocarbon feedstock (1) comprising aromatic compounds containing 9 carbon atoms are isomerized in an isomerization unit (A) in the presence of a bifunctional isomerization catalyst having a hydro/dehydrogenating function and a hydroisomerizing function, to produce an isomerization effluent (10) enriched in trimethylbenzenes. The present invention also relates to a process and a device for the production of aromatic compounds, comprising the process and the device for the conversion of aromatic compounds.

### TECHNICAL FIELD

The invention relates to the conversion of aromatics in the context of producing aromatics for the petrochemical industry (benzene, toluene, para-xylene, ortho-xylene). The aromatic complex (aromatics production device) is supplied with C6 to C10+ feedstocks, and the alkyl aromatics are extracted therefrom and then converted into the desired intermediates. The products of interest are aromatics with 0, 1 or 2 methyls, xylenes having the greatest market value. It is thus worthwhile having methyl groups. Thus, the invention relates to increasing the amount of methyl groups available in the aromatic complex by converting alkyl chains containing more than two carbons, and notably converting aromatics containing 9 carbon atoms, i.e. A9 cut.

### PRIOR ART

Prior art techniques are known for the conversion of A9 cuts, such as dealkylation reactions (loss of two carbons) and hydrogenolysis reactions (loss of one carbon atom).

A dealkylation reaction is a reaction for the substitution, in a molecule, of a hydrogen atom for an alkyl radical.

A hydrodealkylation reaction is a dealkylation reaction in which the removal of the alkyl group from aromatic molecules is performed in the presence of hydrogen. Specifically, it is a terminal cleavage of the alkyl chain “flush” with the nucleus. The catalysis can be of the acid type, notably used on alkyl chains containing two or more carbons but very inefficient for methyls, or of the metal type, notably when it is desired to convert methyls. The conversion of methyls is notably used for reducing the cut point of gasolines for which all the molecules must lose carbons, or for the production of benzene for which the reaction is pushed to the maximum so as to keep only the aromatic nucleus.

A hydrogenolysis reaction is a chemical reaction via which a carbon-carbon or carbon-heteroatom covalent bond is broken down or undergoes lysis by the action of hydrogen. A hydrodealkylation reaction can therefore be considered to be a reaction for hydrogenolysis of the carbon-carbon bond between an alkyl and an aromatic nucleus. On the other hand, a hydrogenolysis reaction also concerns the carbon-carbon bonds internal to the alkyl group containing two or more carbons.

For example, it may be mentioned that ethyltoluenes can be converted into xylenes by hydrogenolysis (see FR 3069244 A1) or into toluene by dealkylation via a reaction mechanism currently used in transalkylation units. Patent application FR 3 069 244 A1 notably relates to a selective hydrogenolysis unit that treats a feedstock rich in aromatic compounds containing more than 8 carbon atoms, and which consists in converting one or more alkyl groups containing at least two carbon atoms (ethyl, propyl, butyl, isopropyl, etc.) attached to a benzene nucleus into one or more methyl groups.

### SUMMARY OF THE INVENTION

In the context described previously, a first object of the present description is to overcome the problems of the prior art and to provide a process for producing aromatics for the petrochemical industry allowing improved methyl compound selectivity and yield.

The present invention relates to the isomerization reaction (no loss of carbon) of aromatics containing 9 carbon atoms bearing alkyl chains containing 2 or 3 carbon atoms. It is therefore a matter of isomerizing the following 5 compounds into trimethylbenzenes (TMB): cumene, n-propylbenzene, o-methylethylbenzene, m-ethyltoluene and p-ethyltoluene, so as to increase the amount of methyl groups available. Advantageously, by transalkylation with toluene, the net xylene and notably p-xylene production of an aromatic complex can then be increased.

According to a first aspect, the abovementioned objects, along with other advantages, are obtained by means of a process for converting aromatic compounds, comprising the following steps:
isomerizing the aromatic compounds (e.g. cumene, n-propylbenzene, o-ethyltoluene, m-ethyltoluene and p-ethyltoluene) of a hydrocarbon feedstock comprising aromatic compounds containing 9 carbon atoms in an isomerization unit in the presence of a bifunctional isomerization catalyst having a hydro/dehydrogenating function and a hydroisomerizing function, to produce an isomerization effluent enriched in trimethylbenzenes.

According to one or more embodiments, the isomerization of the aromatic compounds of the hydrocarbon feedstock is performed under at least one of the following operating conditions:
temperature of between 250° C. and 450° C., preferentially between 355° C. and 390° C., such as a temperature of 385° C.;
pressure of between 0.1 MPa absolute and 3 MPa absolute, preferentially between 0.2 MPa absolute and 1.5 MPa absolute;
H₂/HC mole ratio of between 1 and 5, and preferentially between 3 and 4.5, such as an H₂/HC mole ratio of 4;
WWH of between 1 h⁻¹ and 30 h⁻¹, preferentially between 3 h⁻¹ and 12 h⁻¹, the term WWH corresponding to the weight of hydrocarbon feedstock injected per hour and relative to the weight of catalyst supplied.

According to one or more embodiments, the isomerization catalyst comprises at least one metal from group VIIIB of the Periodic Table of the Elements as hydro/dehydrogenating function, at least one molecular sieve as hydroisomerizing function, and optionally at least one matrix.

According to one or more embodiments, the feedstock comprises aromatic compounds containing 9 carbon atoms bearing alkyl chains containing 2 or 3 carbon atoms, such as cumene, n-propylbenzene, o-methylethylbenzene, m-ethyltoluene and p-ethyltoluene.

According to one or more embodiments, the conversion process comprises the following step:
treating the isomerization effluent in a separation unit located, optionally directly, downstream of the isomerization unit, to produce at least a first separation cut and a cut of unconverted compounds recycled to the inlet of the isomerization unit.

According to one or more embodiments, the conversion process comprises the following step:
treating the hydrocarbon feedstock in an extraction unit located, optionally directly, upstream of the isomerization unit, to extract trimethylbenzenes and produce a trimethylbenzene-depleted hydrocarbon feedstock sent to the isomerization unit.

According to a second aspect, the abovementioned objects, along with other advantages, are obtained by means of a xylene production process incorporating the conversion process according to the first aspect, and comprising the following step:
sending all or some of the isomerization effluent enriched in trimethylbenzenes to an aromatic complex, and preferably to a transalkylation unit, to produce xylenes.

According to one or more embodiments, the process for converting aromatic compounds is integrated into an aromatic complex according to at least one of the following configurations:
pretreatment of the hydrocarbon feedstock upstream of the aromatic complex;
treatment of at least one cut internal to the aromatic complex.

According to one or more embodiments, the xylene production process comprises the following step:
sending an (e.g. essentially) aromatic effluent comprising compounds containing 9 to 10 carbon atoms (C9-C10) from a xylene column of the aromatic complex to the isomerization unit as a hydrocarbon feedstock.

According to a third aspect, the abovementioned objects, along with other advantages, are obtained by means of an aromatic compound conversion device, comprising an isomerization unit suitable for isomerizing the aromatics (e.g. cumene, n-propylbenzene, o-ethyltoluene, m-ethyltoluene and p-ethyltoluene) of a hydrocarbon feedstock comprising aromatic compounds containing 9 carbon atoms, in the presence of a bifunctional isomerization catalyst having a hydro/dehydrogenating function and a hydroisomerizing function, to produce an isomerization effluent enriched in trimethylbenzenes.

According to one or more embodiments, the isomerization catalyst comprises at least one metal from group VIIIB of the Periodic Table of the Elements as hydro/dehydrogenating function, at least one molecular sieve as hydroisomerizing function, and optionally at least one matrix.

According to one or more embodiments, the conversion device comprises:
a separation unit located, optionally directly, downstream of the isomerization unit, suitable for treating the isomerization effluent to produce at least a first separation cut and a cut of unconverted compounds recycled to the inlet of the isomerization unit.

According to one or more embodiments, the conversion device comprises:
an extraction unit located, optionally directly, upstream of the isomerization unit suitable, for treating the hydrocarbon feedstock to extract trimethylbenzenes and produce a trimethylbenzene-depleted hydrocarbon feedstock sent to the isomerization unit.

According to a fourth aspect, the abovementioned objects, along with other advantages, are obtained by means of a xylene production device incorporating the device for converting aromatic compounds according to the third aspect, and comprising:
a feed line suitable for sending all or some of the isomerization effluent enriched in trimethylbenzenes to an aromatic complex, and preferably to a transalkylation unit, to produce xylenes.

According to one or more embodiments, the conversion device is integrated into the aromatic complex according to at least one of the following configurations:
pretreatment of the hydrocarbon feedstock (e.g. part of the input feedstock) upstream of the aromatic complex;
treatment of at least one cut internal to the aromatic complex.

According to one or more embodiments, the xylene production device comprises:
a feed line suitable for sending an (e.g. essentially) aromatic effluent comprising compounds containing 9 to 10 carbon atoms (C9-C10) from a xylene column of the aromatic complex to the isomerization unit as hydrocarbon feedstock.

Embodiments according to the abovementioned aspects and also other characteristics and advantages of the devices and processes according to the abovementioned aspects will become apparent on reading the description which will follow, given solely by way of illustration and without limitation, and with reference to the following drawings.

### LIST OF FIGURES

FIG. 1 represents an aromatic compound conversion device according to one or more embodiments of the present invention, comprising an isomerization unit, an optional trimethylbenzene extraction unit located directly upstream of the isomerization unit, and an optional column for separating products, by-products, reaction intermediates and unconverted species.

FIG. 2 represents an aromatic complex for the production of para-xylene, incorporating an aromatic compound conversion device according to one or more embodiments of the present invention.

### DESCRIPTION OF THE EMBODIMENTS

In the petrochemical industry, para-xylene is one of the intermediates with the highest market value. Its production requires methyl-substituted monoaromatics; it is mainly produced by disproportionation of toluene, isomerization of xylenes or transalkylation of toluene with trimethylbenzenes or tetramethylbenzenes. To maximize the production of para-xylene, it is useful to maximize the amount of methyl group available per aromatic nucleus.

With this aim in mind, methyl-substituted monoaromatics, preferably monoaromatics substituted only with methyls, can be directly upgraded, which is not the case for monoaromatics bearing alkyl chains containing more than two carbons (e.g. ethylbenzene, methylethylbenzenes (MEB), propylbenzenes, etc.). It is therefore preferable to convert these monoaromatics into aromatics (e.g only) substituted with methyls. In this context, a device has been developed for converting aromatic compounds, comprising a unit for the isomerization of aromatic compounds containing 9 carbon atoms, which is capable of increasing the amount of methyl groups on the aromatic nuclei, notably to increase the production of para-xylene. Advantageously, the isomerization unit notably allows the production of trimethylbenzenes from propylbenzenes and methylethylbenzenes.

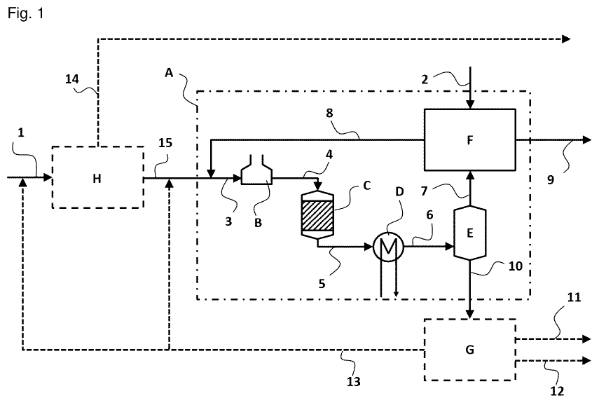

According to the first and third aspects and with reference to FIG. 1, the present invention thus relates to a process and a device for converting aromatic compounds, using/comprising an isomerization unit A suitable for isomerizing the aromatics (e.g. cumene, n-propylbenzene, o-ethyltoluene, m-ethyltoluene and p-ethyltoluene) of a hydrocarbon feedstock 1 comprising aromatic compounds containing 9 carbon atoms, and producing an isomerization effluent enriched in trimethylbenzenes.

### The Hydrocarbon Feedstock

According to one or more embodiments, the hydrocarbon feedstock 1 comprises at least 95% by weight, preferably at least 98% by weight and very preferably at least 99% by weight of aromatics relative to the total weight of said hydrocarbon feedstock 1. According to one or more embodiments, the hydrocarbon feedstock 1 comprises at least 93% by weight, preferably at least 95% by weight and very preferably at least 98% by weight of aromatics comprising at least 9 carbon atoms relative to the total weight of said hydrocarbon feedstock 1.

According to one or more embodiments, the hydrocarbon feedstock 1 comprises at least 50% by weight, preferably at least 60% by weight, preferentially at least 70% by weight, of aromatic molecules comprising at least one C2+ alkyl (e.g. ethyl, propyl) chain relative to the total weight of the hydrocarbon feedstock 1.

According to one or more embodiments, the hydrocarbon feedstock 1 comprises or consists essentially of aromatic compounds containing 9 carbon atoms bearing alkyl chains containing 2 or 3 carbon atoms, such as cumene, n-propylbenzene, o-methylethylbenzene, m-ethyltoluene and p-ethyltoluene.

According to one or more embodiments, the hydrocarbon feedstock 1 comprises at least 93.5% by weight, preferably at least 95.5% by weight and very preferably at least 98.5% by weight of aromatic molecules containing between 9 and 10 carbon atoms relative to the total weight of said hydrocarbon feedstock 1. According to one or more embodiments, the hydrocarbon feedstock comprises at least one internal stream of an aromatic complex for the production of para-xylene and/or the isomerization effluent 10 is a feedstock at least partly sent to an aromatic complex for the production of para-xylene.

According to one or more embodiments, the hydrocarbon feedstock 1 comprises at least 93% by weight, preferably at least 95% by weight and very preferably at least 98% by weight of aromatic molecules containing 9 carbon atoms relative to the total weight of said hydrocarbon feedstock 1. According to one or more embodiments, the hydrocarbon feedstock 1 comprises methylethylbenzenes and/or propylbenzenes and optionally trimethylbenzenes, and preferably little or no (e.g. less than 1% by weight, preferably less than 0.5% by weight, very preferably less than 0.2% by weight) of trimethylbenzenes.

According to one or more embodiments, the hydrocarbon feedstock 1 comprises at least 0.1% by weight, preferably at least 0.2% by weight and very preferably at least 0.5% by weight of aromatic molecules containing 10 carbon atoms relative to the total weight of said hydrocarbon feedstock 1. According to one or more embodiments, the hydrocarbon feedstock 1 comprises dimethylethylbenzenes and/or methylpropylbenzenes and optionally tetramethylbenzenes and/or butylbenzene.

According to one or more embodiments, the hydrocarbon feedstock 1 comprises at least 93% by weight, preferably at least 95% by weight and very preferably at least 98% by weight of aromatic compounds chosen from methylethylbenzenes, propylbenzenes, optionally trimethylbenzenes, dimethylethylbenzenes, methylpropylbenzenes and optionally tetramethylbenzenes and/or butylbenzene.

### The Isomerization Unit

With reference to FIG. 1, the isomerization unit A is suitable for:
treating a hydrocarbon feedstock 1 comprising aromatic compounds containing 9 carbon atoms, by means of a hydrogen supply 2 and in the presence of a catalyst, to convert at least some of the hydrocarbon feedstock 1 into trimethylbenzenes; and to produce a conversion effluent 5 enriched in trimethylbenzenes.

According to one or more embodiments, the isomerization unit A comprises at least one isomerization reactor C suitable for use under the following operating conditions:
temperature of between 250° C. and 450° C., preferentially between 355° C. and 390° C., such as a temperature of 385° C.; and/or
pressure of between 0.1 MPa absolute and 3 MPa absolute, preferentially between 0.2 MPa absolute and 1.5 MPa absolute; and/or
H₂/HC mole ratio of between 1 and 5, and preferentially between 3 and 4.5; such as an H₂/HC mole ratio of 4; and/or
WWH of between 1 h⁻¹ and 30 h⁻¹, preferentially between 3 h⁻¹ and 12 h⁻¹.

The term “WWH” corresponds to the mass of hydrocarbon feedstock injected per hour, relative to the mass of catalyst supplied.

According to one or more embodiments, the isomerization reactor C is a fixed bed or moving bed reactor. A moving bed may be defined as being a gravity flow bed, such as those encountered in the catalytic reforming of gasolines. According to one or more embodiments, the isomerization reactor C is a fixed bed reactor.

According to one or more embodiments, the hydrocarbon feedstock 1 is mixed with the supply of hydrogen 2 in the isomerization reactor C and/or (e.g. directly) upstream of the isomerization reactor C to form a hydrogen-enriched hydrocarbon feedstock 3.

According to one or more embodiments, the isomerization unit A also comprises a heating unit B for heating the hydrocarbon feedstock 1 or the hydrogen-enriched hydrocarbon feedstock 3 (e.g. directly) upstream of the isomerization reactor C. The heating unit B may be preceded by conversion effluent heat recovery equipment 5 used for preheating the hydrocarbon feedstock 1 or the hydrogen-enriched hydrocarbon feedstock 3. According to one or more embodiments, the heating unit B is suitable for use under the following operating conditions: inlet temperature of between 150° C. and 200° C.; and/or outlet temperature of between 355° C. and 390° C. (e.g. 385° C.). The heating effluent 4 from the heating unit B is sent (e.g. directly) to the isomerization reactor C.

According to one or more embodiments, the conversion effluent 5 is sent (e.g. directly) to a cooling unit D (e.g. heat exchanger) to form a cooled conversion effluent 6. The cooling unit D may be preceded by conversion effluent heat recovery equipment 5 used for preheating the hydrocarbon feedstock 1 or the hydrogen-enriched hydrocarbon feedstock 3. According to one or more embodiments, the cooling unit 15 is suitable for use under the following operating conditions: inlet temperature of between 355° C. and 390° C. (e.g. 385° C.); and/or output temperature of between 45° C. and 60° C.

According to one or more embodiments, the cooled conversion effluent 6 is sent (e.g. directly) to a separating section E to produce a gaseous effluent 7 comprising hydrogen and an isomerization effluent 10.

According to one or more embodiments, the gaseous effluent 7 is sent to a recycling unit F suitable for: compressing and/or purifying the gaseous effluent 7; optionally extracting a purge gas 9 (e.g. methane) from the gaseous effluent 7; and/or mixing the gaseous effluent 7 with the hydrogen supply 2 to form a hydrogen mixture 8 sent to the isomerization reactor C and/or (e.g. directly) mixed with the hydrocarbon feedstock 1 to form the hydrogen-enriched hydrocarbon feedstock 3.

### The Separation Unit

With reference to FIG. 1, according to one or more embodiments, the aromatic compounds conversion device also comprises an optional separation unit G optionally located (e.g. directly) downstream of the isomerization unit A, for treating the isomerization effluent 10 and producing at least one separation cut, such as a first separation cut 11 and a second separation cut 12, and optionally an unconverted compounds cut 13 that can be recycled to the inlet of the isomerization unit A.

According to one or more embodiments, the first separation cut 11 is a hydrocarbon cut comprising compounds containing 8 carbon atoms or less (C8−); the second separation cut 12 is an aromatic cut comprising trimethylbenzenes; and the unconverted compounds cut 13 is an aromatic cut comprising methylethylbenzenes and propylbenzenes.

### The Extraction Unit

In order to improve the performance of the isomerization unit A, according to one or more embodiments, it is proposed to add (e.g. directly) upstream of the isomerization unit A, an extraction (or depletion) unit H to extract the trimethylbenzenes and thus reduce the content of compounds (only) substituted with methyls. These compounds do not need to be isomerized prior to transalkylation and therefore do not need to be treated by the isomerization unit A.

Thus, the feedstock of the isomerization unit is depleted in trimethylbenzenes, enabling the isomerization unit A to process predominantly aromatics bearing at least one alkyl chain containing two or more carbons. Thus, the losses in the isomerization unit A are reduced, resulting in a gain in the selectivity of the unit.

With reference to FIG. 1, the conversion device comprises an extraction unit H suitable for:
treating the hydrocarbon feedstock 1 so as to extract trimethylbenzenes; and
producing an effluent 14 enriched in trimethylbenzenes and a hydrocarbon feedstock 15 depleted in trimethylbenzenes, sent to the isomerization unit A instead of the hydrocarbon feedstock 1.

According to one or more embodiments, the effluent 14 enriched in trimethylbenzenes comprises at least 50% by weight, preferably at least 60% by weight and very preferably at least 70% by weight of trimethylbenzenes, relative to the total weight of said effluent.

According to one or more embodiments, the extraction unit H comprises at least one distillation column, and/or a molecular sieve simulated moving bed, and/or a molecular sieve adsorption unit which can be regenerated at temperature and/or under differential pressure, and/or a crystallization unit, and/or a liquid/liquid extraction unit, and/or an extractive distillation unit, and/or a membrane separation unit.

According to one or more embodiments, the extraction unit H comprises at least one of the following distillation columns:
a first extraction column suitable for recovering methylethylbenzenes and/or propylbenzenes at the top of the column and trimethylbenzenes at the bottom of the column.

According to one or more embodiments, the column of the extraction unit H is suitable for use under at least one of the following operating conditions:
reflux vessel having a pressure substantially between 0.001 and 0.1 MPag, such as substantially 0.01 MPag, and a temperature between substantially 140° C. and 180° C., such as substantially 163° C.;
column having substantially from 50 to 150 theoretical plates, such as substantially 100 theoretical plates, a mass ratio of reflux and feed rates of between 1 and 10, preferably between 4 and 6, a temperature at the top of the column of between 150° C. and 190° C., preferably between 160° C. and 175° C., and a temperature at the bottom of the column of between substantially 180° C. and 220° C., such as substantially 203° C.

According to one or more embodiments, when, for example, the extraction unit H is used in combination with the separation unit G, the first separation cut 11 is a head fraction cut comprising compounds containing 8 carbon atoms or less (C8−); the second separation cut 12 is an optional purge cut (e.g. fuel gas); and the unconverted compounds cut 13 is a bottom fraction cut comprising trimethylbenzenes, methylethylbenzenes and propylbenzenes, which is sent to the extraction unit H.

### The Isomerization Catalyst

According to the invention, the isomerization reactor C is operated in the presence of a bifunctional isomerization catalyst, i.e. a hydroisomerization catalyst having a hydro/dehydrogenating function or element and a hydroisomerizing function or element.

In the present patent application, the term “hydro/dehydrogenating” refers to the promotion of a hydro/dehydrogenation reaction which comprises/consists of the incorporation/removal of hydrogen atoms in a molecule. In the present patent application, the term “hydroisomerizing” refers to the promotion of a hydroisomerization reaction which comprises/consists of the transformation of a molecule into an isomer, in the presence of hydrogen.

According to the invention, the hydro/dehydrogenating and hydroisomerizing catalyst comprises at least one metal from group VIIIB of the Periodic Table of the Elements as the hydro/dehydrogenating function or element and at least one molecular sieve as the hydroisomerizing function or element. According to one or more embodiments, the isomerization catalyst also comprises at least one matrix.

In the present patent application, the groups of chemical elements are given, by default, according to the CAS classification (CRC Handbook of Chemistry and Physics, published by CRC Press, Editor-in-Chief D. R. Lide, 81st edition, 2000-2001). For example, group VIIIB according to the CAS classification corresponds to the metals from columns 8, 9 and 10 according to the new IUPAC classification. Groups IIIA, IVA and VIIB according to the CAS classification correspond to the metals of columns 13, 14 and 7 according to the new IUPAC classification, respectively.

### The Hydroisomerizing Element

According to one or more embodiments, the at least one molecular sieve comprises at least one zeolite molecular sieve. According to one or more embodiments, the catalyst comprises at least one one-dimensional 10 MR or 12 MR zeolite molecular sieve. One-dimensional 10 MR or 12 MR zeolite molecular sieves have pores or channels whose aperture is defined by a ring containing 10 oxygen atoms (10 MR aperture) or 12 oxygen atoms (12 MR aperture). The channels of the zeolite molecular sieve having a 10 MR or 12 MR aperture advantageously include non-interconnected one-dimensional channels which open directly to the exterior of said zeolite. According to one or more embodiments, the one-dimensional 10 MR or 12 MR zeolite molecular sieves present in said hydroisomerization catalyst comprise silicon and at least one element T chosen from the group consisting of aluminium, iron, gallium, phosphorus and boron. Preferably the element T comprises or consists of aluminium.

According to one or more embodiments, the one-dimensional 10 MR zeolite molecular sieve of the hydroisomerization catalyst is advantageously chosen from zeolite molecular sieves of the framework type TON (e.g. chosen from ZSM-22 and NU-10, taken alone or as a mixture), FER (e.g. chosen from ZSM-35 and ferrierite, taken alone or as a mixture), EUO (e.g. chosen from EU-1 and ZSM-50, taken alone or as a mixture), AEL (e.g. SAPO-11) or *MRE (e.g. chosen from ZSM-48, ZBM-30, EU-2 and EU-11, taken alone or as a mixture). According to one or more embodiments, the 12 MR zeolite molecular sieve of the hydroisomerization catalyst is chosen from zeolite molecular sieves of the framework type MTW (e.g. chosen from ZSM-12, TPZ-12, Theta-3, NU-13, CZH-5, taken alone or as a mixture) and MOR (e.g. chosen from mordenite or LZ-211, taken alone or as a mixture). The framework codes are defined in the International Zeolite Association classification (IZA: http://www.iza-structure.org/databases/).

According to one or more embodiments, the catalyst comprises an IZM-2 zeolite. IZM-2 zeolite is a crystalline microporous solid having a crystal structure described in patent application FR 2 918 050 A1. IZM-2 zeolite has an X-ray diffraction pattern including at least the lines listed in Table 1 representing the average dₕₖₗ values and relative intensities measured in an X-ray diffraction pattern of the calcined IZM-2 crystalline solid. In table 1, VS=very strong; S=strong; m=medium; mw=moderately weak; w=weak; vw=very weak. The relative intensity Iᵣₑₗ is given as a relative intensity scale in which a value of 100 is attributed to the most intense line in the X-ray diffraction diagram: vw<15; 15≤w<30; 30≤mw<50; 50≤m<65; 65≤S<85; VS≤85.

TABLE 1

| 2 theta (°) | dₕₖₗ (Å) | Iᵣₑₗ |
|---|---|---|
| 5.07 | 17.43 | vw |
| 7.36 | 12.01 | VS |
| 7.67 | 11.52 | VS |
| 8.78 | 10.07 | S |
| 10.02 | 8.82 | vw |
| 12.13 | 7.29 | vw |
| 14.76 | 6.00 | vw |
| 15.31 | 5.78 | vw |
| 15.62 | 5.67 | vw |
| 16.03 | 5.52 | vw |
| 17.60 | 5.03 | vw |
| 18.22 | 4.87 | vw |
| 19.01 | 4.66 | vw |
| 19.52 | 4.54 | vw |
| 21.29 | 4.17 | m |
| 22.44 | 3.96 | w |
| 23.10 | 3.85 | mw |
| 23.57 | 3.77 | w |
| 24.65 | 3.61 | vw |
| 26.78 | 3.33 | w |
| 29.33 | 3.04 | vw |
| 33.06 | 2.71 | vw |
| 36.82 | 2.44 | vw |
| 44.54 | 2.03 | vw |

The diffraction diagram is obtained by radiocrystallographic analysis by means of a diffractometer using the conventional powder method with the Kα₁ radiation of copper (λ=1.5406 Å). On the basis of the position of the diffraction peaks represented by the angle 2θ, the lattice constant distances dₕₖₗ characteristic of the sample are calculated using the Bragg relationship. The measurement error Δ(dₕₖₗ) over dₕₖₗ is calculated by means of Bragg's law as a function of the absolute error Δ(2θ) assigned to the measurement of 2θ. An absolute error Δ(2θ) equal to +0.02° is commonly accepted. The relative intensity Iᵣₑₗ assigned to each value of dₕₖₗ is measured according to the height of the corresponding diffraction peak. The X-ray diffraction diagram of the IZM-2 crystalline solid according to the invention includes at least the lines at the values of dₕₖₗ given in Table 1. In the column of the dₕₖₗ values, the mean values of the lattice spacings have been shown in angstroms (Å). Each of these values must be assigned the measurement error Δ(dₕₖₗ) of between ±0.6 Å and ±0.01 Å.

IZM-2 zeolite has a chemical composition expressed on an anhydrous basis, in terms of moles of oxides, defined by the following general formula: XO₂: aY₂O₃: bM_{2/n}O in which X represents at least one tetravalent element, Y represents at least one trivalent element and M is at least one alkali metal and/or alkaline-earth metal of valency n, a and b respectively representing the number of moles of Y₂O₃ and M_{2/n}O and a is between 0 and 0.5 and b is between 0 and 1.

According to one or more embodiments, X is preferentially chosen from silicon, germanium, titanium and a mixture of at least two of these tetravalent elements. According to one or more embodiments, Y is chosen from aluminium, boron, iron, indium and gallium; preferentially, Y is aluminium.

According to one or more embodiments, the IZM-2 zeolite has a chemical composition expressed on an anhydrous basis, in terms of moles of oxides, defined by the following general formula: SiO₂: a Al₂O₃: b M_{2/n}O, in which M is at least one alkali metal and/or one alkaline-earth metal having a valency n. In said formula given above, a represents the number of moles of Al₂O₃ and b represents the number of moles of M_{2/n}O, and a is between 0 and 0.5 and b is between 0 and 1.

According to one or more embodiments, M is chosen from lithium, sodium, potassium, calcium, magnesium and a mixture of at least two of these metals; preferentially, M is sodium.

The Si/Al ratios of the zeolites described above are advantageously those obtained during synthesis or obtained after post-synthesis dealumination treatments that are well known to those skilled in the art, such as, but non-exhaustively, hydrothermal treatments optionally followed by acid attacks or alternatively direct acid attacks with mineral or organic acid solutions. The zeolites are preferably essentially in acid form, i.e. the atomic ratio between the monovalent compensation cation (for example sodium) and the aluminium inserted into the crystal lattice of the solid is advantageously less than 0.1, preferably less than 0.05 and very preferably less than 0.01. According to one or more embodiments, the zeolites included in the composition of said hydroisomerization catalyst are advantageously calcined. According to one or more embodiments, said zeolites are exchanged by means of at least one treatment with a solution of at least one ammonium salt so as to obtain the ammonium form of the zeolites which, once calcined, leads to the acid form of said zeolites.

According to one or more embodiments, the molecular sieve content in the hydroisomerization catalyst is between 1% by weight and 90% by weight, preferably between 3% by weight and 80% by weight, and more preferentially between 4% by weight and 60% by weight, relative to the total weight of the hydroisomerization catalyst.

### The Matrix

According to one or more embodiments, the matrix is amorphous or crystalline. According to one or more embodiments, the matrix is advantageously chosen from the group formed by alumina, silica, silica-alumina, clays, titanium oxide, boron oxide, zirconia and aluminates, taken alone or as a mixture. Preferably, alumina is used as matrix. Preferably, said matrix may contain alumina in all its forms known to those skilled in the art, for instance aluminas of alpha, gamma, eta and delta type.

According to one or more embodiments, the content of matrix, such as alumina, in the hydroisomerization catalyst is between 10% by weight and 99% by weight relative to the total weight of the hydroisomerization catalyst, i.e. so as to provide the remainder to 100% by weight of the elements constituting the hydroisomerization catalyst.

The catalyst support comprises the molecular sieve optionally as a mixture with the matrix. The forming of the support in the form of a mixture is preferably performed by co-kneading, extrusion and then heat treating the molecular sieve with the matrix or a precursor of the matrix, for instance boehmite, which is transformed into alumina by heat treatment.

### The Hydro/Dehydrogenating Element

According to one or more embodiments, the at least one group VIIIB metal is chosen from iron, cobalt, nickel, ruthenium, rhodium, palladium, osmium, iridium and platinum. Preferably, the at least one group VIIIB metal is chosen from VIIIB noble metals; very preferably, the at least one group VIIIB metal is chosen from palladium and platinum and even more preferably the at least one group VIIIB metal is platinum.

According to one or more embodiments, the dispersion of the at least one group VIIIB metal (percentage of the atoms of said metal exposed on the surface), determined by chemisorption, for example by H₂/O₂ titration or by carbon monoxide chemisorption, is between 10% and 100%, preferably between 20% and 100% and more preferably between 30% and 100%. The macroscopic distribution coefficient of the at least one group VIIIB metal, obtained from its profile determined with a Castaing microprobe, defined as the ratio of the concentrations of the group VIIIB metal at the core of the grain (catalyst extrudate) relative to at the edge of this same grain, is between 0.7 and 1.3 and preferably between 0.8 and 1.2. The value of this ratio, in the region of 1, is evidence of the homogeneity of distribution of the at least one group VIIIB metal in the hydroisomerization catalyst.

According to one or more embodiments, the hydroisomerization catalyst also comprises at least one additional metal chosen from the group formed by metals of groups IIIA, IVA and VIIB of the Periodic Table of Elements, preferably chosen from gallium, indium, tin and rhenium. Said additional metal is preferably chosen from indium, tin and rhenium.

Advantageously, the hydro/dehydrogenating element (metal) may be introduced onto the catalyst support by any method known to those skilled in the art, for instance co-kneading, dry impregnation or impregnation by exchange.

According to one or more embodiments, the content of group VIIIB metal, such as platinum, in the hydroisomerization catalyst is between 0.01% by weight and 4% by weight, preferably between 0.05% by weight and 2% by weight, relative to the total weight of the hydroisomerization catalyst.

According to one or more embodiments, the content of the at least one additional metal in the hydroisomerization catalyst is between 0.01% by weight and 2% by weight, preferably between 0.05% by weight and 1% by weight, relative to the total weight of the hydroisomerization catalyst.

According to one or more embodiments, the sulfur content in the hydroisomerization catalyst is such that the ratio of the number of moles of sulfur to the number of moles of the at least one group VIIIB metal is between 0.3 and 3. According to one or more embodiments, the presence of sulfur in the catalyst results from an optional sulfurization step of the hydroisomerization catalyst. According to one or more embodiments, the presence of sulfur in the catalyst results from impurities that may be present, for instance in the alumina binder.

According to one or more embodiments, the hydroisomerization catalyst used in the process according to the invention more particularly comprises, and preferably consists of:
from 1% to 90%, preferably from 3% to 80% and even more preferably from 4% to 60% by weight of molecular sieves;
from 0.01% to 4% and preferably from 0.05% to 2% by weight of at least one group VIIIB metal, preferably platinum;
optionally from 0.01% to 2% and preferably from 0.05% to 1% by weight of at least one additional metal chosen from the group formed by metals of groups IIIA, IVA and VIIB;
optionally a sulfur content, preferably such that the ratio of the number of moles of sulfur to the number of moles of the group VIIIB metal(s) is between 0.3 and 3; and
optionally at least one matrix, preferably alumina, providing the remainder to 100% in the catalyst, relative to the total weight of the hydroisomerization catalyst.

According to one or more embodiments, the hydroisomerization catalyst is formed into cylindrical or polylobal extrudates such as straight or twisted bilobal, trilobal or polylobal extrudates. According to one or more embodiments, the hydroisomerization catalyst is formed into crushed powders, tablets, rings, beads or wheels. Techniques other than extrusion, such as pelletizing or dredging, may advantageously be used.

In the case where the hydroisomerization catalyst contains at least one noble metal, the noble metal contained in said hydroisomerization catalyst may advantageously be reduced. One of the preferred methods for performing the metal reduction is treatment under hydrogen (e.g. between 0.4 and 40 normal m³ hydrogen/h/m³ catalyst (Nm³/h/m³), and preferably between 1 and 16 Nm³/h/m³, such as substantially 4 Nm³/h/m³) at a temperature of between 150° C. and 650° C. and a total pressure of between 0.1 and 25 MPa. For example, a reduction may comprise a steady stage at 150° C. for two hours, then a rise in temperature up to 450° C. at a rate of 1° C./min and then a steady stage of two hours at 450° C.; during the reduction step, the hydrogen flow rate may be 1000 normal m³ of hydrogen per m³ of catalyst and the total pressure may be kept constant at 0.1 MPa. Any ex-situ reduction method may advantageously be envisaged.

### The Aromatic Complex

According to the second and fourth aspects, the conversion process and device are integrated in an aromatic complex, for example in a process and/or device for the production of xylenes using an aromatic complex. The conversion process/device then exchanges streams with the aromatic complex. According to one or more embodiments, the aromatic complex is fed with hydrocarbon cuts essentially containing molecules whose carbon number ranges from 6 to 10.

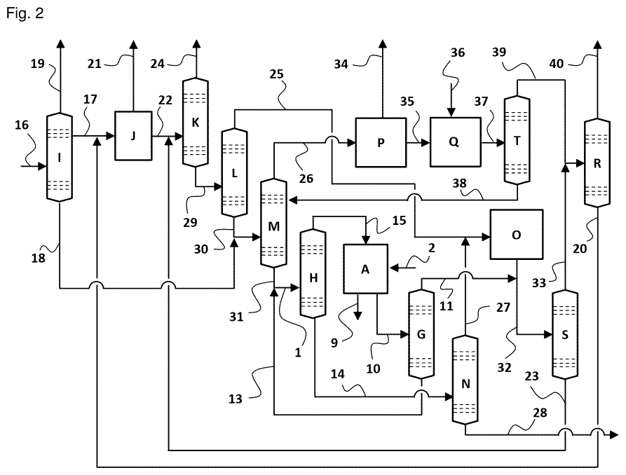

According to one or more embodiments, the following configurations of a conversion device integrated into an aromatic complex are envisioned:
the conversion device is used as a pretreatment unit upstream of the aromatic complex. In this case, external streams can directly feed the conversion device (for example reformate of 6 to 10 carbons, A9/A10 cut, etc.), and the effluents from the conversion device are then sent to the aromatic complex;
one or more conversion devices are used to treat one or more cuts internal to the aromatic complex. In this case, the conversion device can be partially or totally fed with one or more streams coming from units (e.g. fractionation/distillation columns, simulated moving bed) of the aromatic complex. The effluents from the conversion device are then also returned to the aromatic complex;
the combination of the two configurations defined above is also possible and remains within the context of the present invention. In all cases, the effluents are then enriched in aromatics comprising methyl groups which are totally or partially sent to the aromatic complex in order to produce xylenes and optionally benzene. Overall, as will be shown in the embodiment of FIG. 2 described below, the integration of the conversion device into the aromatic complex increases the production of para-xylene.

According to one or more embodiments, the conversion device is suitable for treating a stream containing aromatics containing 9 carbon atoms and optionally 10 carbon atoms internal to the aromatic complex. For example, FIG. 2 shows a conversion device integrated into an aromatic complex for treating a stream containing aromatics containing 9 and 10 carbon atoms obtained from the fractionation train of the aromatic complex.

With reference to FIG. 2, according to one or more embodiments, the aromatic complex comprises:
an optional feedstock separation unit I for separating a hydrocarbon cut containing 7 carbon atoms or less (C7−) and an aromatic cut containing 8 carbon atoms or more (A8+) from the feedstock entering the aromatic complex;
an optional unit J for extraction of the aromatics between the feedstock separation unit I and a fractionation train K-N in order to separate the aliphatic compounds from the benzene and toluene of the C7-cut of the feedstock of the complex;
the fractionation train K-N making it possible to extract the xylenes from the other aromatics;
a transalkylation unit O which converts toluene and methylalkylbenzenes such as trimethylbenzenes into xylenes; advantageously, this unit can also treat tetramethylbenzenes and, to a certain extent, benzene;
a xylene separation unit P (e.g. crystallization unit or simulated moving bed separation unit using a molecular sieve and a desorbent) or a unit of the type that enables the para-xylene to be isolated from the xylenes and the ethylbenzene;
an optional unit Q for isomerizing the raffinate obtained as effluent from the xylene separation unit P, to convert in particular ortho-xylene, meta-xylene and ethylbenzene into para-xylene; and
a conversion device according to the present invention comprising an isomerization unit A, a separation unit G and an extraction unit H which are suitable for treating a hydrocarbon feedstock 1 produced at the bottom of the xylene column M of the aromatic complex; and for producing an isomerization effluent 10.

According to one or more embodiments, the feedstock separation unit I treats the feedstock 16 entering the aromatic complex, to separate a head fraction cut 17 comprising (e.g. essentially) compounds containing 7 carbon atoms or less (C7−), and a bottom fraction cut 18 comprising (e.g. essentially) aromatics containing 8 carbon atoms or more (A8+), which is sent to the xylene column M. Optionally, the feedstock separation unit I can also separate a light compound cut 19 (compounds comprising 5 carbon atoms or less).

According to one or more embodiments, the entering feedstock 16 is a hydrocarbon cut predominantly containing molecules whose carbon number ranges from 6 to 10 carbon atoms. This feedstock may also contain molecules containing more than 10 carbon atoms and/or molecules containing 5 carbon atoms. The feedstock 16 entering the aromatic complex is rich in aromatics and contains at least 50% by weight of alkyl aromatics, preferentially more than 70% by weight. The entering feedstock 16 can be produced by catalytic reforming of a naphtha or can be a product of a cracking (e.g. steam cracking, catalytic cracking) unit or any other means for the production of alkyl aromatics.

The head fraction cut 17 from the feedstock separation unit I, optionally mixed with the bottom product 20 (benzene and toluene) from a stabilization column R, is sent to the aromatics extraction unit J so as to extract an effluent 21 comprising C6-C7 aliphatic species 21, which is exported as a co-product of the aromatic complex. The aromatic cut 22 (essentially benzene and toluene) called the extract from the aromatics extraction unit J, optionally mixed with the heavy fraction 23 from the (first) separation column S of the transalkylation unit O, is sent to the (first) aromatic compound distillation column K of the fractionation train K-N.

According to one or more embodiments, the fractionation train comprises the columns for the distillation of aromatic compounds K, L, M and N, making it possible to separate the following five cuts:
a cut 24 comprising (e.g. essentially) aromatic compounds containing 6 carbon atoms (e.g. benzene);
a cut 25 comprising (e.g. essentially) aromatic compounds containing 7 carbon atoms (e.g. toluene);
a cut 26 comprising (e.g. essentially) aromatic compounds containing 8 carbon atoms (e.g. xylenes and ethylbenzene);
a cut 27 comprising (e.g. essentially) monoaromatic compounds containing 9 and 10 carbon atoms; and
a cut 28 comprising (e.g. essentially) aromatic compounds, the most volatile species of which are aromatics containing 10 carbon atoms.

The first column K for distilling aromatic compounds, also referred to as the benzene column, is suitable for: treating a C6-C10 (e.g. essentially) aromatic (A6+) hydrocarbon feedstock 22; producing at the top the cut 24 (benzene cut) which is one of the desired products exiting the aromatic complex; and producing at the bottom a C7-C10 (e.g. essentially) aromatic (A7+) effluent 29. According to one or more embodiments, the C6-C10 (e.g. essentially) aromatic (A6+) hydrocarbon feedstock 22 is a C6-C7 (e.g. essentially) aromatic (A6-A7) hydrocarbon feedstock.

The second column L for distilling aromatic compounds, also referred to as the toluene column, is suitable for: treating the (A7+) effluent 29 from the bottom of the benzene column; producing at the top the cut 25 (toluene cut) which is sent to the transalkylation unit O; and producing at the bottom a C8-C10 (e.g. essentially) aromatic (A8+) effluent 30.

The third column M for distilling aromatic compounds, also referred to as the xylene column, is suitable for: treating the effluent 30 from the bottom of the toluene column and optionally an aromatic cut 18 containing 8 or more carbon atoms (A8+) of the feedstock of the aromatic complex; producing at the top the cut 26 (xylenes and ethylbenzene cut) which is sent to the xylene separation unit P; and producing at the bottom a C9-C10 (e.g. essentially) aromatic (A9+) effluent 31 as hydrocarbon feedstock 1 for the conversion device according to the invention.

The fourth aromatic compound distillation column N, also known as the heavy aromatics column, is suitable for: treating the trimethylbenzene-enriched effluent 14 from the extraction unit H; producing at the top the cut 27 comprising (e.g. essentially) monoaromatic compounds containing 9 and 10 carbon atoms which is directed to the transalkylation unit O; and producing at the bottom the cut 28 comprising (e.g. essentially) aromatic compounds of which the most volatile species are aromatics containing 10 carbon atoms (A10+).

In the transalkylation unit O, the cut 27 comprising (e.g. essentially) monoaromatic compounds containing 9 and 10 carbon atoms is mixed with the cut 25 comprising toluene coming from the top of the toluene column L, to produce xylenes by transalkylation of aromatics lacking methyl groups (toluene), and having an excess of methyl groups (e.g. trimethylbenzenes and tetramethylbenzenes), and is fed to the first separation column S. According to one or more embodiments, the transalkylation unit O is fed with benzene (line not shown in FIG. 2), for example when an excess of methyl groups is observed for the production of para-xylene.

According to one or more embodiments, the transalkylation unit O comprises at least a first transalkylation reactor suitable for use under at least one of the following operating conditions:
temperature of between 200° C. and 600° C., preferentially between 350° C. and 550° C. and even more preferentially between 380° C. and 500° C.;
pressure of between 2 and 10 MPa, preferentially between 2 and 6 MPa and more preferentially between 2 and 4 MPa;
WWH of between 0.5 and 5 h⁻¹, preferentially between 1 and 4 h⁻¹, and more preferentially between 2 and 3 h⁻¹.

According to one or more embodiments, the first transalkylation reactor is operated in the presence of a catalyst comprising zeolite, for example ZSM-12 and/or ZSM-5. According to one or more embodiments, the second transalkylation reactor is of fixed bed type.

According to one or more embodiments, the transalkylation unit O comprises at least a second transalkylation reactor suitable for use under at least one of the following operating conditions:
temperature of between 200° C. and 400° C., preferentially between 220° C. and 350° C. and even more preferentially between 250° C. and 310° C.;
pressure of between 1 and 6 MPa, preferentially of between 2 and 5 MPa, and more preferentially of between 3 and 5 MPa;
WWH of between 0.5 and 5 h⁻¹, preferentially between 0.5 and 4 h⁻¹, and more preferentially between 0.5 and 3 h⁻¹.

According to one or more embodiments, the second transalkylation reactor is operated in the presence of a catalyst comprising zeolite, for example dealuminated zeolite Y (for example, a zeolite similar to those described in the alkylation catalyst part). According to one or more embodiments, the second transalkylation reactor is of fixed bed type.

According to one or more embodiments, the transalkylation effluents 32 from the reaction section of the transalkylation unit O are separated in the first separation column S. A cut 33 comprising at least some of the benzene and the more volatile species (C6−) is extracted at the top of the first separation column and is sent to the optional stabilization column R. The heavy fraction 23 of the effluent from the first separation column S comprising (e.g. essentially) aromatics containing at least 7 carbon atoms (A7+) is optionally recycled to the fractionation train K-N, for example to the benzene column K.

The cut 26 comprising (e.g. essentially) aromatic compounds containing 8 carbon atoms (e.g. xylenes and ethylbenzene) is treated in the xylene separation unit P. para-Xylene 34 is exported as the main product. The raffinate 35 from the xylene separation unit P comprising (e.g. essentially) ortho-xylene, meta-xylene and ethylbenzene feeds the isomerization unit Q.

In the isomerization reaction section (not shown) of the isomerization unit Q, the para-xylene isomers can be isomerized while ethylbenzene is dealkylated to produce benzene, in the presence of hydrogen (e.g. fed with a hydrogen source 36). In this example, the isomerization reaction section is of the dealkylating type. According to one or more embodiments, at least one isomerization reaction section of the isomerization unit is of the isomerizing type or the ethylbenzene is isomerized to xylenes. According to one or more embodiments, the isomerization effluents 37 from the isomerization reaction section are sent to a second separation column T to produce, at the bottom, a para-xylene-enriched isomerate 38, which is optionally recycled to the xylene column M; and to produce, at the top, a hydrocarbon cut 39 comprising compounds containing 7 carbon atoms or less (C7−), which is sent to the stabilization column R, for example with the cut 33 comprising at least some of the benzene, and the more volatile species.

According to one or more embodiments, at least one isomerization reaction section is in the gas phase and is suitable for use under at least one of the following operating conditions:
temperature of greater than 300° C., preferably from 350° C. to 480° C.;
pressure of less than 4.0 MPa, and preferably from 0.5 to 2.0 MPa;
hourly space velocity of less than 10 h⁻¹ (10 litres per litre per hour), preferably between 0.5 h⁻¹ and 6 h⁻¹;
hydrogen to hydrocarbon mole ratio of less than 10, and preferably of between 3 and 6;
in the presence of a catalyst including at least one zeolite having pores whose aperture is defined by a ring containing 10 or 12 oxygen atoms (10 MR or 12 MR), and at least one group VIIIB metal in a content of between 0.1% and 0.3% by weight (reduced form), limits included.

According to one or more embodiments, at least one isomerization reaction section is in the liquid phase and is suitable for use under at least one of the following operating conditions:
temperature of less than 300° C., preferably 200° C. to 260° C.;
pressure of less than 4 MPa, preferably 2 to 3 MPa;
hourly space velocity (HSV) of less than 10 h⁻¹ (10 litres per litre per hour), preferably between 2 and 4 h⁻¹;
in the presence of a catalyst including at least one zeolite having pores whose aperture is defined by a ring containing 10 or 12 oxygen atoms (10 MR or 12 MR), preferentially a catalyst including at least one zeolite having pores whose aperture is defined by a ring containing 10 oxygen atoms (10 MR), and even more preferably a catalyst including a zeolite of ZSM-5 type.

According to one or more embodiments, the stabilization column R produces at the bottom a stabilized cut 20 comprising (e.g. essentially) benzene and toluene optionally recycled to the inlet of the aromatics extraction unit J. The stabilization column R notably makes it possible to extract compounds containing 5 carbon atoms or less 40 referred to hereinbelow as combustible gas or fuel gas.

The example of FIG. 2 described above relates to an embodiment in which the conversion device according to the invention is suitable for treating a stream containing aromatics containing 9 and 10 carbon atoms resulting from the fractionating train of the aromatic complex. It should be noted that other configurations, alone or in combinations, are also envisioned.

### EXAMPLES

### Preparation of a Catalyst a Comprising IZM-2 Zeolite

Catalyst A is a catalyst comprising an IZM-2 zeolite, platinum and an alumina matrix.

### Synthesis of the IZM-2 Zeolite

The IZM-2 zeolite was synthesized in accordance with the teaching of patent FR 2 918 050 B1. A colloidal silica suspension known under the trade name Ludox HS-40, sold by Aldrich, is incorporated into a solution composed of sodium hydroxide (Prolabo), 1,6-bis(methylpiperidinium) hexane dibromide structuring agent, sodium aluminate (Carlo Erba) and deionized water. The molar composition of the mixture is as follows: 1 SiO₂; 0.0042 Al₂O₃; 0.1666 Na₂O; 0.1666 1,6-bis(methylpiperidinium) hexane; 33.3333 H₂O. The mixture is stirred vigorously for 30 minutes. The mixture is then transferred, after homogenization, into a Parr autoclave. The autoclave is heated for 5 days at 170° C. with spindle stirring (30 rpm). The product obtained is filtered, washed with deionized water to reach neutral pH and then dried overnight at 100° C. in an oven. The solid is then introduced into a muffle furnace and calcined so as to remove the structuring agent. The calcination cycle comprises a temperature rise up to 200° C., a steady stage of two hours at this temperature, a temperature rise up to 550° C., followed by a steady stage of eight hours at this temperature and finally a return to ambient temperature. The temperature rises are performed at a rate of 2° C./minute. The solid thus obtained is then refluxed for 2 hours in an aqueous ammonium nitrate solution (10 ml of solution per gram of solid, ammonium nitrate concentration of 3M) so as to exchange the sodium alkaline cations with ammonium ions. This refluxing step is performed six times with fresh ammonium nitrate solution, and the solid is then filtered off, washed with deionized water and dried in an oven overnight at 100° C. Finally, to obtain the zeolite in its acid (protonated H+) form, a step of calcination is performed at 550° C. for 10 hours (temperature increase rate of 2° C./minute) in a fluidized bed under dry air (2 normal litres per hour and per gram of solid). The solid thus obtained was analysed by X-ray diffraction and identified as consisting of IZM-2 zeolite. Characterizations by means of X-ray fluorescence (in particular by bead assay on a PANalytical AXIOS machine working at 125 mA and 32 kV) and ICP (in particular on a SPECTRO ARCOS ICP-OES machine according to the ASTM D7260 method) gives access to the following results for IZM-2:
ratio of the number of moles of silicon divided by the number of moles of aluminium, in mol/mol, Si/Al: 85,
ratio of the number of moles of sodium divided by the number of moles of aluminium, in mol/mol, Na/Al: 0.03.

### Forming of the Support

The IZM-2 zeolite is blended with an alumina gel of GA7001 type supplied by the company Axens. The blended paste is extruded through a 1.6 mm diameter cylindrical die. After drying in an oven overnight at 110° C., the extrudates are calcined at 550° C. for two hours (temperature increase rate of 5° C./min) in a fluidized bed under dry air (2 normal litres per hour and per gram of solid). The amount of zeolite used is chosen so as to obtain about 14% by weight of zeolite in the extrudates after calcination.

### Impregnation of the Platinum

The platinum is then deposited in the extrudates by dry impregnation in a dredger with an aqueous solution of platinum tetrammine chloride Pt(NH₃)₄Cl₂. The platinum content in the impregnation solution is adjusted so as to obtain about 0.3% platinum by weight on the catalyst after calcination. After impregnation, the extrudates are left to mature for five hours in the laboratory air and are then dried overnight in an oven at 110° C. The extrudates are then calcined under a flow of dry air in a fluidized bed (1 normal litre per hour and per gram of solid) under the following conditions:
temperature rise from ambient temperature to 150° C. at 5° C./min,
steady stage of 1 hour at 150° C.,
rise from 150° C. to 450° C. at 5° C./min,
steady stage of 1 hour at 450° C.,
decrease to ambient temperature.

Characterizations by X-ray fluorescence, Castaing microprobe and H₂/O₂ titration give access to the following results for catalyst A:
percentage of IZM-2 zeolite (dry mass): 13% by weight,
percentage of platinum (dry mass): 0.31% by weight,
dispersion of the platinum: 66%,
coefficient of distribution of the platinum: 0.85.

### Preparation of a Catalyst B Comprising ZSM-12 Zeolite

Catalyst B is a catalyst comprising a ZSM-12 zeolite, platinum and an alumina matrix.

### Synthesis of the ZSM-12 Zeolite

ZSM-12 zeolite is a commercial zeolite supplied by the company Zeolyst. Its commercial reference is CP788. It is supplied in its ammonium form. The solid thus obtained was analysed by X-ray diffraction and identified as consisting of ZSM-12 zeolite.

### Forming of the Support

The ZSM-12 zeolite is blended with an alumina gel of GA7001 type supplied by the company Axens. The blended paste is extruded through a 1.6 mm diameter cylindrical die. After drying in an oven overnight at 110° C., the extrudates are calcined at 550° C. for two hours (temperature increase rate of 5° C./min) in a fluidized bed under dry air (2 normal litres per hour and per gram of solid). The amount of zeolite used is chosen so as to obtain about 8% by weight of zeolite in the extrudates after calcination.

### Impregnation of the Platinum

The platinum is then deposited in the extrudates by dry impregnation in a dredger with an aqueous solution of platinum tetrammine chloride Pt(NH₃)₄Cl₂. The platinum content in the impregnation solution is adjusted so as to obtain about 0.25% platinum by weight on the catalyst after calcination. After impregnation, the extrudates are left to mature for five hours in the laboratory air and are then dried overnight in an oven at 110° C. The extrudates are then calcined under a flow of dry air in a fluidized bed (1 normal litre per hour and per gram of solid) under the following conditions:
temperature rise from ambient temperature to 150° C. at 5° C./min,
steady stage of 1 hour at 150° C.,
rise from 150° C. to 450° C. at 5° C./min,
steady stage of 1 hour at 450° C.,
decrease to ambient temperature.

Characterizations by X-ray fluorescence, Castaing microprobe and H₂/O₂ titration give access to the following results for catalyst B:
percentage of ZSM-12 zeolite (dry mass): 8% by weight,
percentage of platinum (dry mass): 0.24% by weight,
dispersion of the platinum: 90%,
coefficient of distribution of the platinum: 1.01.

### Preparation of a Catalyst C Comprising EU-1 Zeolite

Catalyst C is a catalyst comprising an EU-1 zeolite, platinum and an alumina matrix.

### Synthesis of the EU-1 Zeolite

An EU-1 zeolite is synthesized according to the teaching of patent EP 0 042 226 B1 using the organic structuring agent 1,6-N,N,N,N′,N′,N′-hexamethylhexamethylenediammonium. For the preparation of such a zeolite, the reaction mixture has the following molar composition: 60 SiO₂: 10.6 Na₂O: 5.27 NaBr: 1.5 Al₂O₃: 19.5 Hexa-Br₂: 2777 H. Hexa-Br₂ is 1,6-N,N, N,N′,N′,N′-hexamethylhexamethylenediammonium, bromine being the counterion. The reaction mixture is placed in an autoclave with stirring (300 rpm) for 5 days at 180° C.

The EU-1 zeolite is first subjected to dry calcination at 550° C. under a flow of dry air for 10 hours to remove the organic structuring agent. The solid obtained is then refluxed for 4 hours in an ammonium nitrate solution (100 ml of solution per gram of solid, ammonium nitrate concentration of 10M) so as to exchange the alkali metal cations with ammonium ions. This exchange step is performed four times. The solid is then calcined at 550° C. for 4 hours in a tube furnace. X-ray diffraction analysis confirms that the EU-1 zeolite has been obtained. Characterizations by means of X-ray fluorescence (in particular by bead assay on a PANalytical AXIOS machine working at 125 mA and 32 kV) and ICP (in particular on a SPECTRO ARCOS ICP-OES machine according to the ASTM D7260 method) give access to the following results for EU-1:
ratio of the number of moles of silicon divided by the number of moles of aluminium, in mol/mol, Si/Al: 15,
ratio of the number of moles of sodium divided by the number of moles of aluminium, in mol/mol, Na/Al: 0.01.

### Forming of the Support

The EU-1 zeolite is blended with an alumina gel of GA7001 type supplied by the company Axens. The blended paste is extruded through a 1.6 mm diameter cylindrical die. After drying in an oven overnight at 110° C., the extrudates are calcined at 550° C. for two hours (temperature increase rate of 5° C./min) in a fluidized bed under dry air (2 normal litres per hour and per gram of solid). The amount of zeolite used is chosen so as to obtain about 10% by weight of zeolite in the extrudates after calcination.

### Impregnation of the Platinum

The support thus obtained is subjected to an anionic exchange with hexachloroplatinic acid in the presence of a competitive agent (hydrochloric acid), so as to deposit 0.3% by weight of platinum relative to the catalyst. The wet solid is then dried at 120° C. for 12 hours and calcined in air at a temperature of 500° C. for one hour.

Characterizations by X-ray fluorescence, Castaing microprobe and H₂/O₂ titration give access to the following results for catalyst C:
percentage of EU-1 zeolite (dry mass): 11% by weight,
percentage of platinum (dry mass): 0.29% by weight,
dispersion of the platinum: 85%,
coefficient of distribution of the platinum: 0.97.

### Example 1

Example 1 illustrates the performance of an isomerization unit A in which an aromatic cut mainly containing 9 carbon atoms is treated, the mass composition of which cut is described in detail in Table 2 below.

TABLE 2

| A6-A7 (weight %) | | | 0.03 |
|---|---|---|---|
| A8 (weight %) | | | 3.23 |
| | A9 (weight %) | Propylbenzene | 8.45 |
| | | Methylethylbenzene | 63.53 |
| | | Trimethylbenzene | 23.87 |
| | | Total A9 | 95.85 |

| | |
|---|---|
| A10 (weight %) | 0.11 |
| Non-aromatic (weight %) | 0.78 |

Once prepared, these catalysts undergo an in situ activation step in the isomerization unit. The catalysts first undergo a drying step under a flow of nitrogen under the following conditions:
Nitrogen flow rate: 5 NI/h/gram of catalyst;
Total pressure: 1.3 MPa absolute;
Temperature increase rate from ambient temperature to 150° C.: 10° C./min;
steady stage of 30 minutes at 150° C.

The nitrogen is then replaced with hydrogen and the catalyst reduction step is performed under the following conditions:
Hydrogen flow rate: 4 NI/h/gram of catalyst;
Total pressure: 1.3 MPa absolute;
Temperature increase rate from 150 to 480° C.: 5° C./min;
steady stage of two hours at 480° C.

After reduction, the temperature is lowered to 425° C. and the catalysts are then stabilized for 24 hours under a flow of A9 feedstock and hydrogen under the following conditions before the catalytic performance is assessed:
Total pressure: 1.3 MPa absolute;
Temperature of the reactor: 385° C.;
Hydrogen coverage: 4 mol of H₂ per mole of hydrocarbons;
WWH: 5 grams of hydrocarbon per gram of catalyst and per hour.

The A9 isomerization unit operates in a fixed bed under the following conditions:
Pressure of the reactor: 1.3 MPa;
Temperature of the reactor: 385° C.;
Hydrogen coverage: 4 mol of H₂ per mole of hydrocarbons;
WWH: 4.5 h⁻¹.

The performance of the test for the three types of catalysts is shown in Table 3. The 5-12% gain in trimethylbenzene shows the advantage of the A9 isomerization unit as described in the present invention.

TABLE 3

| | | Outlet | | |
|---|---|---|---|---|
| | | Catalyst A | Catalyst B | Catalyst C |
| | Inlet | IZM-2 | ZSM-12 | EU-1 |
| | Composition | Composition | Composition | Composition |
| — | (weight %): | (weight %): | (weight %): | (weight %): |
| A8 | 3.23 | 3.40 | 3.22 | 3.08 |
| A9 | 95.85 | 75.39 | 80.47 | 78.12 |
| Propylbenzene | 8.45 | 1.46 | 2.27 | 2.21 |
| Methylethylbenzene | 63.53 | 37.87 | 43.19 | 46.59 |
| Trimethylbenzene | 23.87 | 36.06 | 35.01 | 29.32 |
| A10 | 0.11 | 0.61 | 0.53 | 0.36 |
| Total A8 + A9 + A10 | 99.19 | 154.79 | 84.22 | 81.56 |

| | | | |
|---|---|---|---|
| Gain in TMB (weight %) | 12 | 11 | 5 |
| MEB conversion (weight %) | 40 | 32 | 27 |
| A9 paraffin losses (weight %) | 10 | 5 | 7 |
| A9 losses of NOAs (weight %) | 7 | 5 | 6 |
| Total losses of A9 (weight %) | 17 | 10 | 13 |

NOA: Naphthenes and aromatics other than A9

### Example 2

Example 2 illustrates the performance of an isomerization unit A using catalyst B based on ZSM-12 in combination with an extraction unit H treating an aromatic cut mainly containing 9 carbon atoms. The test performance is presented in Table 4 below.

TABLE 4

| | Line 1 | Line 14 | Line 4 | Line 5 |
|---|---|---|---|---|
| | Composition | Composition | Composition | Composition |
| — | (weight %) | (weight %) | (weight %) | (weight %) |
| Propylbenzene | 5.47 | 0.13 | 13.75 | 8.27 |
| Methylethyl- | 26.81 | 4.41 | 61.59 | 41.93 |
| benzene | | | | |
| Trimethyl- | 50.36 | 74.52 | 12.85 | 29.09 |
| benzene | | | | |
| Total A9 | 82.64 | 79.05 | 88.19 | 79.29 |

| | |
|---|---|
| A9 losses in the isomerization reactor (weight %) | 10% |
| MEB conversion (weight %) | 32% |
| Gain in TMB (weight %) | 16% |

Advantageously, the performance of the conversion device according to the invention is improved with trimethylbenzene depletion of the feedstock by adding a step for extracting the methyl-substituted aromatics. This extraction step is performed by the extraction unit H.

### Example 3

Example 3 illustrates a case (see FIG. 2) in which the conversion device according to the invention treats a cut mainly containing A9 internal to the aromatic complex, since said cut is rich in trimethylbenzene isomers (notably methylethylbenzene).

Specifically, the (e.g. essentially) aromatic C9-C10 effluent 31 (A9+) recovered from the bottom of the xylene column M is sent to the extraction unit H as the hydrocarbon feedstock 1 of the conversion device according to the invention.

The extraction unit H treats the hydrocarbon feedstock 1 to extract trimethylbenzenes and thus produce a trimethylbenzene-enriched effluent 14 and a trimethylbenzene-depleted hydrocarbon feedstock 15 sent to the isomerization unit A.

The trimethylbenzene-enriched effluent 14 (also comprising A10+ compounds) is sent to the heavy aromatics column N which feeds the transalkylation unit O.

The isomerization unit A according to the present invention may be seen as a unit for pretreatment of the A9 cut upstream of the transalkylation unit O.

The isomerization unit A produces an isomerization effluent 10 which may contain xylenes which are extracted by the separation unit G before being fed to the transalkylation unit O. Indeed, the isomerization unit A may be in thermodynamic equilibrium and produce xylenes by A9+/A7 transalkylation. It is thus preferable to extract the xylenes so as not to penalize the conversion.

The inlet feedstock 16 (reformate) feeding the complex has the composition shown in Table 5 below. The total mass flow rate of aromatics is 250 t/h.

TABLE 5

| | |
|---|---|
| Benzene (weight %) | 7.8% |
| Toluene (weight %) | 29.2% |
| A8 (weight %) | 36.4% |
| Trimethylbenzene (weight %) | 11.0% |
| Other A9 (weight %) | 9.8% |
| Tetramethylbenzene (weight %) | 5.7% |

The performance of the aromatic complex with the conversion device according to the invention is shown in Table 6 below.

TABLE 6

| Feedstocks | Reference (t/h) | Invention (t/h) |
|---|---|---|
| Reformate (line 16) | 250.00 | 250.00 |
| H₂ supply (line 36) | 0.97 | 0.97 |
| H₂ supply (line 2) | — | 0.1 |
| Total | 250.97 | 251.07 |

| Products | Reference (t/h) | Invention (t/h) | Delta (t/h) |
|---|---|---|---|
| para-Xylene (line 34) | 145.01 | 154.20 | 9.19 |
| Benzene (line 24) | 53.01 | 43.52 | −9.49 |
| para-Xylene + benzene | 198.02 | 197.72 | −0.30 |
| Raffinate (line 21) | 32.13 | 32.24 | 0.11 |
| C5- cut (line 19) | 3.49 | 3.59 | 0.10 |
| Fuel gas (line 40) | 13.46 | 13.61 | 0.15 |
| Heavy fraction (line 28) | 3.87 | 3.91 | 0.04 |
| Total | 250.97 | 251.07 | 0.10 |

The conversion device according to the invention coupled with the aromatic complex allows in Example 3 a gain in para-xylene production of the order of 6% with an iso-production of para-xylene and benzene.

In the present patent application, the term “to comprise” is synonymous with (means the same thing as) “to include” and “to contain”, and is inclusive or open and does not exclude other elements which are not stated. It is understood that the term “comprise” includes the exclusive and closed term “consist”. In addition, in the present description, the terms “approximately”, “substantially”, “essentially”, “solely” and “about” are synonymous with (mean the same thing as) a margin of greater and/or less than 5%, preferably 2%, very preferably 1%, of the given value. For example, an effluent essentially or solely comprising compounds A corresponds to an effluent comprising at least 95%, preferably at least 98%, very preferably at least 99%, of compounds A. As another example, a value of substantially 100 (° C., MPag, h⁻¹, etc.) corresponds to a value between 95-105, preferably between 98-102, very preferably between 99-101.

1. A process for converting aromatic compounds, comprising:
isomerizing aromatic compounds of a hydrocarbon feedstock (1) comprising aromatic compounds containing 9 carbon atoms in an isomerization unit (A) in the presence of a bifunctional isomerization catalyst having a hydro/dehydrogenating function and a hydroisomerizing function, to produce an isomerization effluent (10) enriched in trimethylbenzenes.

2. The process according to claim 1, wherein the isomerization of the aromatic compounds of the hydrocarbon feedstock (1) is performed under at least one of the following operating conditions:
a temperature of between 250° C. and 450° C.;
a pressure of between 0.1 MPa absolute and 3 MPa absolute;
an H2/HC mole ratio of between 1 and 5;
a WWH of between 1 h-1 and 30 h-1, wherein WWH corresponds to weight of hydrocarbon feedstock injected per hour and relative to weight of catalyst supplied.

3. The process according to claim 1, wherein the isomerization catalyst comprises at least one metal from group VIIIB of the Periodic Table of the Elements as hydro/dehydrogenating function, at least one molecular sieve as hydroisomerizing function, and optionally at least one matrix.

4. The process according to claim 1, wherein the hydrocarbon feedstock (1) comprises aromatic compounds containing 9 carbon atoms bearing alkyl chains containing 2 or 3 carbon atoms.

5. The process according to claim 1, further comprising:
treating the isomerization effluent (10) in a separation unit (G) located, optionally directly, downstream of the isomerization unit (A), to produce at least a first separation cut (11) and a cut of unconverted compounds (13) recycled to the inlet of the isomerization unit (A).

6. The process according to claim 1, further comprising:
treating the hydrocarbon feedstock (1) in an extraction unit (H) located, optionally directly, upstream of the isomerization unit (A), to extract trimethylbenzenes and produce a trimethylbenzene-depleted hydrocarbon feedstock (15) sent to the isomerization unit (A).

7. A process for the production of xylenes comprising:
converting aromatic compounds according to claim 1, and
sending all or some of the isomerization effluent (10) enriched in trimethylbenzenes to an aromatic complex to produce xylenes.

8. The process according to claim 7, wherein the converting aromatic compounds is integrated into an aromatic complex according to at least one of the following configurations:
pretreatment of the hydrocarbon feedstock (1) upstream of the aromatic complex;
treatment of at least one cut internal to the aromatic complex.

9. The process according to claim 8, further comprising:
sending an aromatic effluent comprising compounds containing 9 to 10 carbon atoms from a xylene column (M) of the aromatic complex to the isomerization unit (A) as a hydrocarbon feedstock (1).

10. A device for converting aromatic compounds, comprising:
an isomerization unit (A) suitable for isomerizing the aromatic compounds of a hydrocarbon feedstock (1) comprising aromatic compounds containing 9 carbon atoms, in the presence of a bifunctional isomerization catalyst having a hydro/dehydrogenating function and a hydroisomerizing function, to produce an isomerization effluent (10) enriched in trimethylbenzenes.

11. The device according to claim 10, further comprising:
a separation unit (G) located, optionally directly, downstream of the isomerization unit (A), suitable for treating the isomerization effluent (10) to produce at least a first separation cut (11) and a cut of unconverted compounds (13) recycled to the inlet of the isomerization unit (A).

12. The device according to claim 10, further comprising:
an extraction unit (H) located, optionally directly, upstream of the isomerization unit (A), suitable for treating the hydrocarbon feedstock (1) to extract trimethylbenzenes and produce a trimethylbenzene-depleted hydrocarbon feedstock (15) sent to the isomerization unit (A).

13. A xylene production device comprising:
the conversion device according to claim 10, and
a feed line suitable for sending all or some of the isomerization effluent (10) enriched in trimethylbenzenes to an aromatic complex, to produce xylenes.

14. The xylene production device according to claim 13, wherein the conversion device is integrated into an aromatic complex according to at least one of the following configurations:
pretreatment of the hydrocarbon feedstock (1) upstream of the aromatic complex;
treatment of at least one cut internal to the aromatic complex.

15. The xylene production device according to claim 14, further comprising:
a feed line suitable for sending an aromatic effluent comprising compounds containing 9 to 10 carbon atoms from a xylene column (M) of the aromatic complex to the isomerization unit (A) as hydrocarbon feedstock (1).

16. The process according to claim 1, wherein isomerization of the aromatic compounds of the hydrocarbon feedstock (1) is performed under at least one of the following operating conditions:
a temperature between 355° C. and 390° C.;
a pressure of between 0.2 MPa absolute and 1.5 MPa absolute;
an H2/HC mole ratio of between 3 and 4.5;
a WWH of between 3 h-1 and 12 h-1, wherein WWH corresponds to weight of hydrocarbon feedstock injected per hour and relative to weight of catalyst supplied.
